(19) United States
(12) Patent Application Publication
(10) Pub. No.: US 2009/0023953 A1
(43) Pub. Date: Jan. 22, 2009

(54) PENTAFLUOROSULFANYL CONTAINING AMINO ACIDS

(75) Inventor: John T. WELCH, Albany, NY (US)

Correspondence Address: HESLIN ROTHENBERG FARLEY & MESITI PC, 5 COLUMBIA CIRCLE, ALBANY, NY, 12203, US

(73) Assignee: The Research Foundation of State University of New York, Albany, NY (US)

(21) Appl. No.: 12/033,545

(22) Filed: Feb. 19, 2008

Related U.S. Application Data
(60) Provisional application No. 60/890,253, filed on Feb. 16, 2007.

(51) Int. Cl. C07C 229/30

(52) U.S. Cl. 562/556

(57) ABSTRACT

There are provided SF₅-containing amino acids of the general formulas

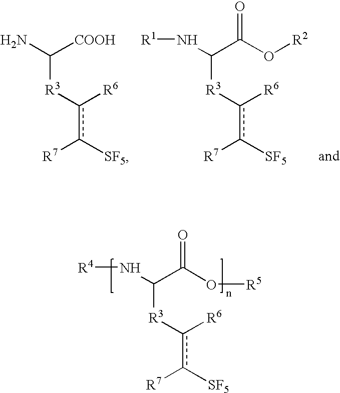

and processes for preparing same. Other embodiments are also disclosed.

### RELATED APPLICATIONS

This application claims priority from U.S. Provisional Application Ser. No. 60/890,253, filed Feb. 16, 2007, the contents of which are incorporated herein by reference.

### FIELD OF THE INVENTION

The present invention relates generally to modified amino acids and processes for preparing same.

### BACKGROUND OF THE INVENTION

Pentafluorosulfanyl groups have only recently been explored as substituents in medicinal and pharmaceutical chemistry. An analog of the trifluoromethyl group (CF₃), the pentafluorosulfanyl group presents an octahedral steric demand while retaining the relative stability of the trifluoromethyl group. The preparation of analogs of biologically active trifluoromethylated compounds requires the ready availability of pentafluorosulfanyl containing reagents and building blocks, materials which only recently have become available.

The development of synthetic methodologies for the introduction of pentafluorosulfanyl groups into organic compounds has been pursued with a considerable degree of interest. SF₅ groups impart unique properties to organic compounds that include low surface energy, high chemical resistance, high thermal stability, high electronegativity, hydrophobicity, and a high dielectric constant. The high electronegativity value of the SF₅ group, 3.62 on the Pauling scale, and its greater electron withdrawing ability than CF₃, make it an attractive alternative to the trifluoromethyl group found in many commercial products.

Various ways of introducing SF₅ into unsaturated organic compounds are known. U.S. Pat. Pub. No. 2006/0069285 discloses a 3-step process for making pentafluorosulfanylnapthalene. U.S. Pat. No. 6,870,068 discloses the addition of SF₅ to organic compounds by condensing gaseous SF₅Br into pentane at a low temperature and reacting in the presence of a free radical initiator. The disclosures of these applications are incorporated herein by reference as they relate to the invention.

The manipulation of SF₅Br is complex, requiring several special apparatuses, including steel bomb tubes, FEP lines or metal lines, and a pressure gauge. Additionally, SF₅Br has to be vacuum-transferred to the reaction vessel. The use of these apparatuses creates significant limitations.

### BRIEF DESCRIPTION OF THE INVENTION

There is provided, in accordance with an embodiment of the invention, a process for preparing SF₅-containing compounds using a stock solution of SF₅Br or SF₅Cl in (a) one or more C₁-C₃ chlorofluorocarbons (b) one or more C₅-C₆ alkanes or (c) a mixture of one or more C₁-C₃ chlorofluorocarbons and one or more C₅-C₆ alkanes. In some embodiments, the chlorofluorocarbon is CCl₃F.

There are also provided, in accordance with embodiments of the invention, amino acids containing SF₅ and processes for preparing such amino acids.

There is also provided, in accordance with an embodiment of the invention, a composition made up of a solution of SF₅Br or SF₅Cl in a solvent selected from (a) one or more C₁-C₃ chlorofluorocarbons, (b) one or more C₅-C₆ alkanes and (c) a mixture of one or more C₁-C₃ chlorofluorocarbons and one or more C₅-C₆ alkanes. In some embodiments, the chlorofluorocarbon is CCl₃F.

There are also provided, in accordance with embodiments of the invention, compounds of formula I, II, and III and salts thereof:

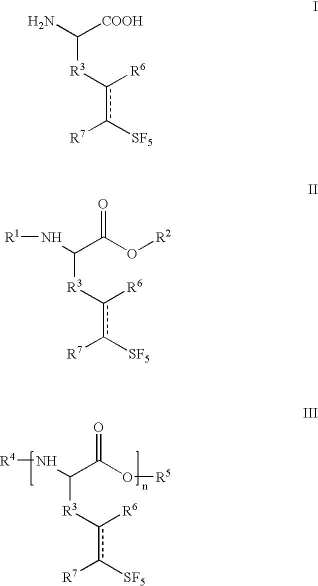

wherein

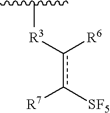

denotes a moiety selected from

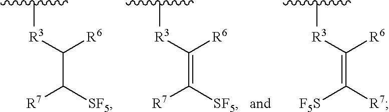

R¹ is chosen from H and an amino protecting group; R² is chosen from H and C₁-C₆ alkyl; R³ is at each occurrence independently chosen from C₁-C₆ alkylene and a direct bond; R⁴ is chosen from H, an amino protecting group, an amino acid, a peptide, and a protein; R⁵ is chosen from H, C₁-C₆ alkyl, an amino acid, a peptide, and a protein; R⁶ is at each occurrence independently chosen from H and C₁-C₂ alkyl, and R⁷ is at each occurrence independently chosen from H and C₁-C₃ alkyl; and n is an integer of 1 or more. In some embodiments, n is 1-50. In some embodiments, n is 1-20. In some embodiments, n is 1, 2, 3, 4, or 5. When R⁵ is a peptide or protein, it may contain additional SF₅-containing amino acids, i.e. there may be multiple SF₅ residues in the polymer that are not contiguous.

There is also provided in accordance with another embodiment of the invention a compound of formula Ia, Ia, or IIIa:

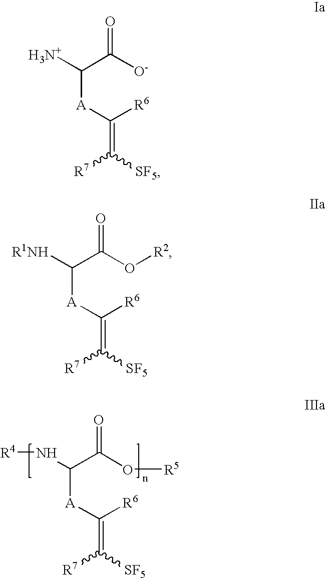

wherein R¹ is chosen from H and an amino protecting group; R² is chosen from H and C₁-C₆ alkyl; A is at each occurrence independently chosen from C₁-C₅ hydrocarbon and a direct bond; R⁴ is chosen from H, an amino protecting group, an amino acid, a peptide, and a protein; R⁵ is chosen from H, C₁-C₆ alkyl, an amino acid, a peptide, and a protein; R⁶ is at each occurrence independently chosen from H and C₁-C₂ alkyl; R⁷ is at each occurrence independently chosen from H and C₁-C₃ alkyl; and n is an integer of 1 or more.

There is also provided, in accordance with an embodiment of the invention, a compound of formula IVa or IVb:

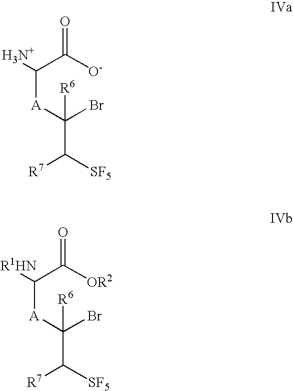

wherein R¹ is chosen from H and an amino protecting group; R² is chosen from H and C₁-C₆ alkyl; A is chosen from C₁-C₅ hydrocarbon and a direct bond; R⁶ is chosen from H and C₁-C₂ alkyl; and R⁷ is chosen from H and C₁-C₃ alkyl.

There is also provided, in accordance with an embodiment of the invention, a process for SF₅X addition wherein X is Cl or Br comprising reacting a compound having an alkene functional group —CH═CH— with a solution of SF₅X in a solvent selected from (a) one or more C₁-C₃ chlorofluorocarbons, (b) one or more C₅-C₆ alkanes and (c) a mixture of one or more C₁-C₃ chlorofluorocarbons and one or more C₅-C₆ alkanes, where said reacting yields an SF₅X addition product

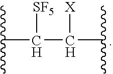

There is also provided, in accordance with another embodiment of the invention, a process for preparing a compound of formula IV

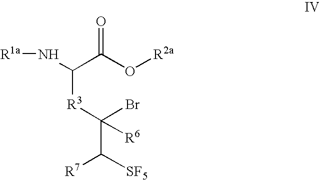

wherein R^{1a} is an amino protecting group, R^{2a} is C₁-C₆ alkyl, R³ is chosen from C₁-C₆ alkylene and a direct bond; R⁶ is chosen from H and C₁-C₂ alkyl; and R⁷ is chosen from H and C₁-C₃ alkyl, comprising reacting a compound of formula V

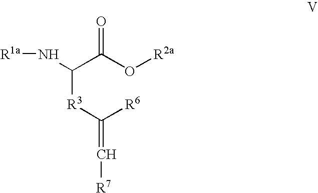

wherein R^{1a}, R^{2a}, R³, R⁶ and R⁷ are as defined above, with a solution of SF₅Br in a solvent selected from (a) one or more C₁-C₃ chlorofluorocarbons, (b) one or more C₅-C₆ alkanes and (c) a mixture of one or more C₁-C₃ chlorofluorocarbons and one or more C₅-C₆ alkanes to form a compound of Formula IV. In some embodiments, the C₁-C₃ chlorofluorocarbon solvent is CCl₃F.

There is also provided, in accordance with an embodiment of the invention, a process for preparing a compound of formula VI

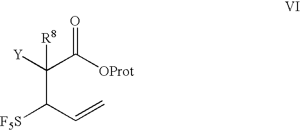

wherein R⁸ is H or alkyl, Prot is an enol protecting group, and Y is a protected amine, comprising reacting a compound of formula VII

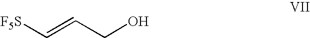

with a compound of formula VIII

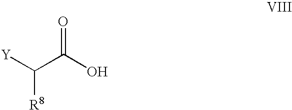

to obtain a compound of formula IX

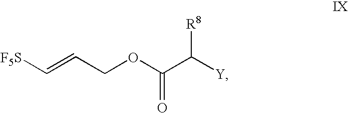

then reacting the compound formula IX with a strong, non-nucleophilic base, followed by reacting the resulting mixture with an enolate-reactive reagent for protecting an enol to obtain a compound of formula X

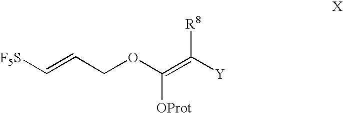

and exposing the compound of formula X to conditions in which the compound of formula X rearranges to form a compound of formula VI. In some embodiments, R⁸ is C₁-C₆ alkyl.

There is also provided, in accordance with embodiments of the invention, a compound of formula VI per se, as well as compounds VIa, VIb, VIc and VId:

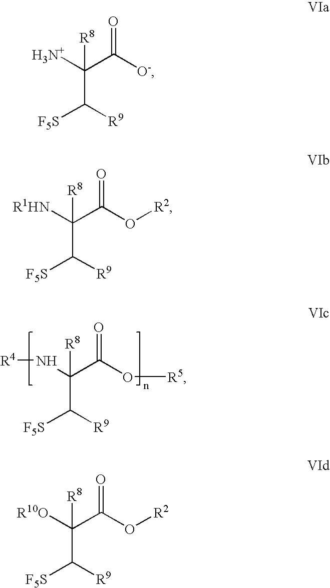

wherein R¹ is chosen from H and an amino protecting group; R² is chosen from H and C₁-C₆ alkyl; R⁸ is independently at each occurrence chosen from H and C₁-C₃ alkyl; R⁹ is independently at each occurrence chosen from —CH═CH₂ and —CH₂CH₃; and R¹⁰ is chosen from H and an alcohol protecting group. In some embodiments, in the compound of formula VI, Y is NHBoc and Prot is trialkyl silyl.

### DETAILED DESCRIPTION OF EMBODIMENTS OF THE INVENTION

Solutions of SF₅X in one or more C₁-C₃ chlorofluorocarbons, one or more C₅-C₆ alkanes, or a mixture of at least one C₁-C₃ chlorofluorocarbon and at least one C₅-C₆ alkanes may be used to prepare SF₅ containing compounds, including SF₅-containing amino acids. Such SF₅-containing amino acids may have utility as tools to construct coiled-coil receptors for RNA and DNA therapies based upon inhibition of RNA or DNA function, suppressing undesirable biological activities such as overproduction of endogenous proteins or enzymes. SF₅ containing amino acids can reasonably be anticipated to have a broad general therapeutic utility as an adjuvant or conjugate in combination with other more hydrophobic chemotherapeutic agents. As an example, SF₅ containing amino acids may have utility when conjugated to pharmaceutical agents such as docetaxel or paclitaxel by improving bioavailablity and active form. The processes disclosed herein for preparing such SF₅-containing amino acids, which use the SF₅X solutions disclosed herein, may be carried out using normal glassware under inert atmosphere such as argon gas. The stock solutions of SF₅X in C₁-C₃ chlorofluorocarbon have been observed to be stable for a substantial amount of time, generally for at least four months in the dark at 4° C. The use of stock solutions of SF₅X avoids problems associated with the use of vacuum lines. Furthermore, it has been found that use of such solutions enables the addition of SF₅X to olefins over a range of temperatures, e.g. at −78° C., 0° C. and room temperature.

Throughout this specification the terms and substituents retain their definitions.

For convenience and clarity, certain terms that are employed in the specification, examples, and claims are described herein.

Alkyl is intended to include linear, branched, or cyclic hydrocarbon structures and combinations thereof. Lower alkyl refers to alkyl groups of from 1 to 6 carbon atoms. Examples of lower alkyl groups include methyl, ethyl, propyl, isopropyl, butyl, s- and t-butyl and the like. Preferred alkyl and alkylene groups are those of C₂₀ or below.

The term strong non-nucleophilic base refers to a non-nucleophilic basic reagent, which does not act as a nucleophile or bind to the reagents utilized according to the reaction. A number of non-nucleophilic bases are known in the art and include sodium hydride, potassium hydride, lithium diisopropylamide and potassium hexamethyldisilazide. In general, a strong non-nucleophilic base has an apparent pKₐ of 19 or greater.

The term enolate-reactive reagent for protecting an enol refers to a reagent that reacts with an enolate to form an ether. A list of examples, including trimethylclorosilane (TMSCl), can be found in Green and Wuts, “Protective Groups in Organic Synthesis” third edition, pg. 113-148 (1999), incorporated herein by reference. “Prot” refers to an enol protecting group. Trialkyl silyl groups are particularly well suited as enol protecting groups.

It will be recognized that the compounds in accordance with embodiments of this invention can exist in radiolabeled form, i.e., the compounds may contain one or more atoms containing an atomic mass or mass number different from the atomic mass or mass number usually found in nature. Radioisotopes of hydrogen, carbon, phosphorous, fluorine, and chlorine include ²H, ³H, ¹³C, ¹⁴C, ¹⁵N, ³⁵S, ¹⁸F, and ³⁶Cl, respectively.

As used herein, and as would be understood by the person of skill in the art, the recitation of “a compound” is intended to include salts, solvates, co-crystals and inclusion complexes of that compound.

The compounds described herein may contain asymmetric centers and may thus give rise to enantiomers, diastereomers, and other stereoisomeric forms. Each chiral center may be defined, in terms of absolute stereochemistry, as (R)- or (S)-. The present invention is meant to include all such possible isomers, as well as, their racemic and optically pure forms. Optically active (R)- and (S)-isomers may be prepared using chiral synthons or chiral reagents, or resolved using conventional techniques. The prefix “rac” refers to a racemate. When the compounds described herein contain olefinic double bonds or other centers of geometric asymmetry, it is intended that the compounds include both E and Z geometric isomers, unless specified otherwise or unless olefinic stereochemistry other than that shown would not yield the desired reaction product. The representation of the configuration of any carbon-carbon double bond appearing herein is selected for convenience only, and unless explicitly stated, is not intended to designate a particular configuration. Thus a carbon-carbon double bond depicted arbitrarily as E may be Z, E, or a mixture of the two in any proportion. Likewise, all tautomeric forms are also intended to be included. For example, formula V

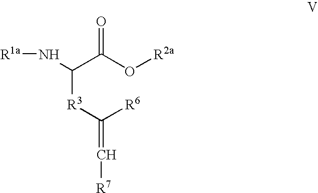

implies either or both

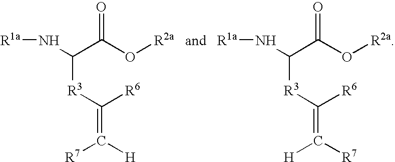

The graphic representations of racemic, ambiscalemic and scalemic or enantiomerically pure compounds used herein are taken from Maehr J. Chem. Ed. 62, 114-120 (1985): solid and broken wedges are used to denote the absolute configuration of a chiral element; wavy lines and single thin lines indicate disavowal of any stereochemical implication which the bond it represents could generate; solid and broken bold lines are geometric descriptors indicating the relative configuration shown but denoting racemic character; and wedge outlines and dotted or broken lines denote enantiomerically pure compounds of indeterminate absolute configuration.

Terminology related to “protecting”, “deprotecting” and “protected” functionalities occurs throughout this application. Such terminology is well understood by persons of skill in the art and is used in the context of processes that involve sequential treatment with a series of reagents. In that context, a protecting group refers to a group, which is used to mask a functionality during a process step in which it would otherwise react, but in which reaction is undesirable. The protecting group prevents reaction at that step, but may be subsequently removed to expose the original functionality. The removal or “deprotection” occurs after the completion of the reaction or reactions in which the functionality would interfere. Thus, when a sequence of reagents is specified, as it is in the processes of the invention, the person of ordinary skill can readily envision those groups that would be suitable as “protecting groups”. Suitable groups for that purpose are discussed in standard textbooks in the field of chemistry, such as Protective Groups in Organic Synthesis by Greene and Wuts [John Wiley & Sons, New York, 1999], which is incorporated herein by reference. Common protecting groups for the amine function of an amino acid include tBoc, CBZ, and Fmoc.

A comprehensive list of abbreviations utilized by organic chemists appears in the first issue of each volume of the Journal of Organic Chemistry. The list, which is typically presented in a table entitled “Standard List of Abbreviations”, is incorporated herein by reference.

The term chlorofluorocarbon refers to haloalkanes with both chlorine and fluorine. Hydrochlorofluorocarbons (HCFCs) represents a class of haloalkanes where not all hydrogen has been replaced by chlorine or fluorine and constitute a subset of chlorofluorocarbons C₅₋alkane and C₆₋alkane refer to alkanes having 5 or 6 carbon atoms, respectively, and may be straight- or branched-chain, cyclic, or cyclic with straight or branch components, e.g. n-pentane, 2-methylbutane, n-hexane, 3-methylpentane, 2-cyclopropylpropyl, dimethylcyclopropyl and the like.

The term peptide refers to two or more amino acids covalently joined by peptide bonds. The term protein, as used herein, refers to polypeptides of 10 kDa molecular weight or more. Further, temperature does not appear to be a critical variable.

As stated, in some embodiments there are provided solutions of SF₅X in a solvent selected from (a) one or more C₁-C₃ chlorofluorocarbons, (b) one or more C₅-C₆ alkyls, and (c) a mixture of at least one C₁-C₃ chlorofluorocarbon and at least one C₅-C₆ alkyls where X is bromine or chlorine. In some embodiment the concentration is 0.01-1.0 M of SF₅ in the solvent. In some embodiments the concentration is 0.4-0.9 M of SF₅ in the solvent.

It will be appreciated that although amino acids, e.g. compounds of formulae I and Ia, may be depicted in the un-ionized form or the zwitterionic form, such compounds may exist in either form, and both forms are intended.

In some embodiments, R⁴ in the compound of formula III or IIIa is t-butoxycarbonyl. In some embodiments, R³ in the compound of formula I, Ia, II, IIa, III, or IIIa is chosen from a direct bond, —CH₂—, —CH(CH₃)—, —CH(CH₃)—CH₂—, and —CH₂—CH(CH₃)—.

### EXAMPLES

### Preparation of Stock Solutions of SF₅Cl and SF₅Br in Hexane

Into 40 mL of distilled, dried hexane was condensed 10.0 g to 13.2 g of SF₅Br with suitable care taken to protect the system from air and moisture. Several repetitions of this procedure resulted in solutions of 1.2-1.6 M. It was found that such solutions could be stored at 4° C. under a nitrogen atomosphere for many months without degradation of the SF₅Br. The solutions were easily transferred for use in the reactions described below by using standard syringe techniques.

Similarly, for SF₅Cl, into 30 mL of distilled dried hexane was condensed 3.3 g to 3.7 g of SF₅Cl with suitable care taken to protect the system from air and moisture. Several repetitions of this procedure resulted in solutions of 0.68-0.75 M. It was found that such solutions could be stored at −20° C. under a nitrogen atomosphere for many months without degradation of the SF₅Cl. Such cold solutions were employed in the reactions described below, utilizing standard syringe techniques.

By analogous procedures, stock solutions of SF₅Br and SF₅Cl in CCl₃F were prepared.

### Experimental Example 1

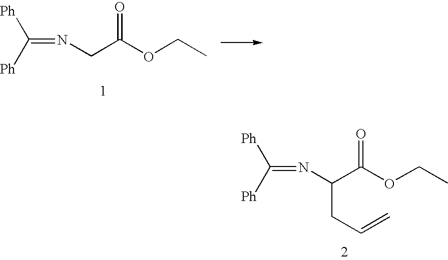

To a solution of diisopropyl amine (1.05 eq, 2.20 ml, 15.7 mmol) in THF (30 mL) was added n-BuLi (1.05 eq, 6.28 mL, 15.7 mmol) slowly at 0° C. then stirred for 30 min. Schiff base 1 (1 eq, 4 g, 15.0 mmol) was added to the reaction mixture at −78° C., which was then stirred for 1 h, followed by addition of allyl bromide (1.05 eq, 1.90 g, 15.7 mmol). After stirring for an additional hour, the temperature of the reaction mixture was allowed to slowly increase to 0° C. then the reaction mixture was extracted with ethyl acetate and water. The organic layer was separated and the aqueous layer washed with ethyl acetate. The combined organic layers were dried over MgSO₄ and then concentrated. The product 2 was obtained in 95% crude yield (4.37 g).

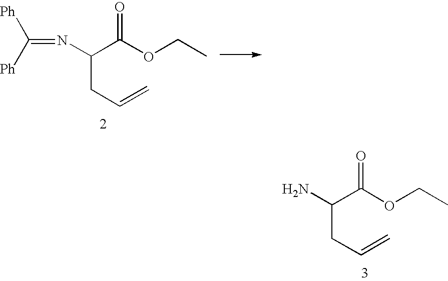

A 15% solution of citric acid (40 mL) was added to the monoalkylated Schiff base 2 (2.2 g, 7.2 mmol) in THF (40 mL) at room temperature. The reaction mixture was then was allowed to stir for 11 h. Acid-base work up with 10% HCl, sat. NaHCO₃ solution and dichloromethane gave the crude product 3 in 70% yield (0.77 g). The purity without further purification was sufficient to proceed to the next step.

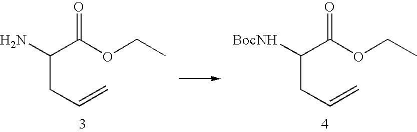

To a solution of the amine compound 3 (1 eq, 2.2 g, 15.4 mmol) in dichloromethane (30 mL) was added Et₃N (3 eq, 4.66 g, 46.1 mmol) and di-tert-butyl dicarbonate (1.2 eq, 4.04 g, 18.5 mmol) at room temperature. The reaction mixture was then allowed to stir for 3 h. The reaction was quenched with water then extracted with dichloromethane and water. The combined organic layers were dried over MgSO₄ and concentrated. The crude product 4 was purified by chromatography according to Still to afford 3.07 g of a colorless liquid product (82% yield).

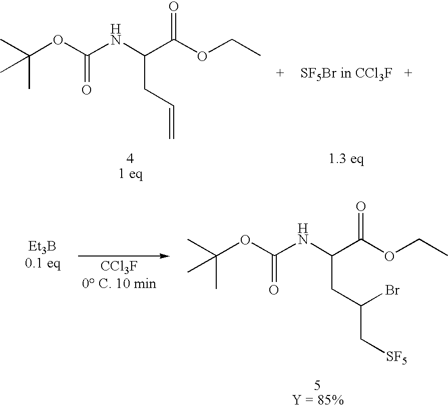

To 7.8 ml of a 0.55 M solution of pentafluorosulfanyl bromide (0.89 g, 4.3 mmol) in CCl₃F was added a 1 M solution of triethylborane in hexane (0.1 eq, 0.33 mL, 0.33 mmol) followed by the slow addition of Boc-protected amino acid 4 (1 eq, 0.80 g, 3.3 mmol) at 0° C. The reaction mixture was stirred for 10 min at 0° C. The reaction was quenched with sat. NaHCO₃ solution, then extracted with dichloromethane and water. The combined organic layers were dried over MgSO₄ and concentrated. The crude product was purified by chromatograph according to Still to afford 1.26 g of 5 as a colorless liquid (85% yield).

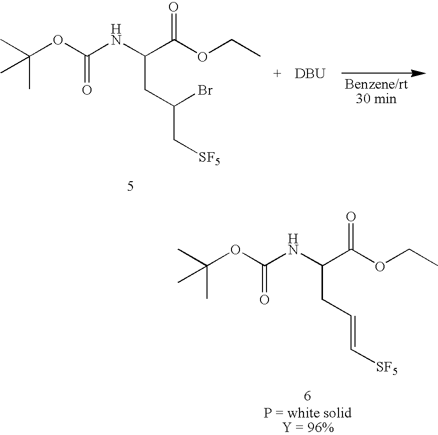

To a solution of bromide compound 5 (1 eq, 1.25 g, 2.78 mmol) in benzene (10 mL) was added diazabicyclo[5.4.0]undec-7-ene (DBU) (1.2 eq, 0.507 g, 3.33 mmol). The reaction mixture was then stirred for 30 min at room temperature. The reaction was quenched with water, then extracted with dichloromethane and water. The combined organic layers were dried over MgSO₄ and then concentrated. The crude product was purified by chromatograph according to Still to afford 0.99 g of 6 as a colorless liquid (96% yield).

### Experimental Example 2

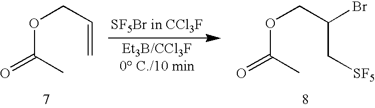

To 4.5 ml of a 0.87 M solution of pentafluorosulfanyl bromide (0.81 g, 3.9 mmol) in CCl₃F was added an 1 M solution of triethylborane in hexane (0.1 eq, 0.30 mL, 0.30 mmol) followed by the slow addition of allyl acetate (1 eq, 0.30 g, 3.0 mmol) at 0° C. The reaction mixture was stirred for 10 min at 0° C. The reaction was quenched with sat. NaHCO₃ solution, then extracted with dichloromethane and water. The combined organic layers were dried over MgSO₄ and concentrated. The crude product was purified by kügelrohr distillation (9 mmHg at 74-76° C.) to afford 0.91 g of 8 as a colorless liquid (99% yield).

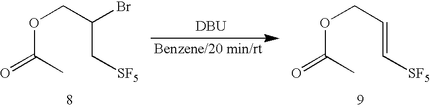

To a solution of bromide compound 8 (1 eq, 0.40 g, 1.30 mmol) in benzene (7 mL) was added DBU (1.2 eq, 0.238 g, 1.56 mmol). The reaction mixture was then stirred for 20 min at room temperature. The reaction was quenched with water, then extracted with hexane and water. The combined organic layers were dried over MgSO₄ and then concentrated. The crude product was purified by flash chromatograph to afford 0.268 g of 9 as a water-white liquid (91% yield).

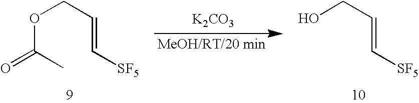

To a solution of 3-pentafluorosufanylallyl acetate 9 (1 eq, 1.40 g, 6.19 mmol) in wet methanol (8 mL) was added potassium carbonate (3 eq, 2.57 g, 18.6 mmol). The reaction mixture was then stirred for 10 min at room temperature. After removal of methanol, the reaction mixture was extracted with dichloromethane and water. The combined organic layers were dried over MgSO₄ and then concentrated. The crude product was purified by flash chromatograph to afford 0.845 g of 10 as a colorless liquid (74% yield).

Compounds of the formula VIa, VIb, VIc and VId in which R⁹ is —CH═CH₂ can be made by the following reaction scheme:

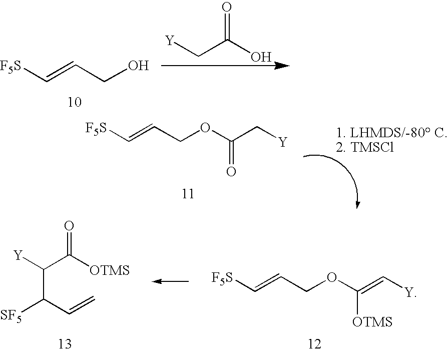

As will be appreciated by those skilled in the art, when R⁹ is —CH₂CH₃, an additional reduction step is necessary. Conditions are well known by a person of ordinary skill in the art. [See, e.g. Burke, S. D.; Ng, R. A.; Morrison, J. A.; Albedi, M. J. Tandem Glycolate Claisen Rearrangement/Ring-Closing Metathesis: A Stereochemically General Synthesis of Substituted Dihydropyran-2-carboxylates J. Org. Chem. 1998, 63, 3160-61. Miller, J. F.; Ternir, F.; Koch, K.; Picopio, A. D. Stereoselective Synthesis of Functionalized Carbocycles and Heterocycles via an Ester Enolate Claisen/Ring-Closing Metathesis Manifold J. Org. Chem., 1998, 63, 3158-59. K. Araki, J. T. Welch, “An Improved Procedure for the Ireland-Claisen Rearrangement of Fluoroacetates,” Tetrahedron Lett. 1993, 34, 2251-2254. K. Araki, W. Q. Yun, J. O'Toole, P. Toscano and J. T. Welch, Carbohydr. Res. 1993, 249, 139-161. “The Synthesis of 2,3-Dideoxy-2-Fluoro-3-C-Methyl-pentose Containing Nucleosides via [3,3] Sigmatropic Rearrangements.”]

The present invention is not limited to the compounds found in the above examples, and other compounds falling within the scope of the invention may also be prepared using the procedures set forth in the above synthetic schemes. The preparation of such compounds using these methods will be apparent to one of ordinary skill in the chemical arts.

The invention has been described in detail with particular reference to some embodiments thereof, but it will be understood by those skilled in the art that variations and modifications can be effected within the spirit and scope of the invention.

1. A compound selected from the group consisting of formulae I-I, II-II, and III-III: 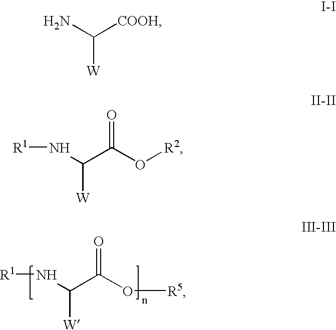
or a salt thereof,
wherein
W is selected from 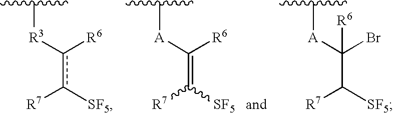
W′ is selected from 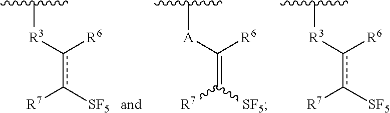
denotes a moiety selected from 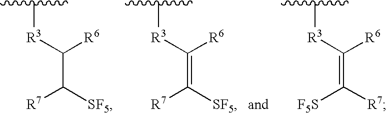
R¹ is chosen from H and an amino protecting group;
R² is chosen from H and C₁-C₆ alkyl;
R³ is independently at each occurrence chosen from C₁-C₆ alkylene and a direct bond;
A is independently at each occurrence chosen from C₁-C₅ hydrocarbon and a direct bond;
R⁴ is chosen from H, an amino protecting group, an amino acid, a peptide, and a protein;
R⁵ is chosen from H, C₁-C₆ alkyl, an amino acid, a peptide, and a protein;
R⁶ is independently at each occurrence chosen from H and C₁-C₂ alkyl and
R⁷ is independently at each occurrence chosen from H and C₁-C₃ alkyl, and
n is an integer of 1 or more.

2. A compound according to claim 1 which is selected from the group consisting of formulae I, II and III: 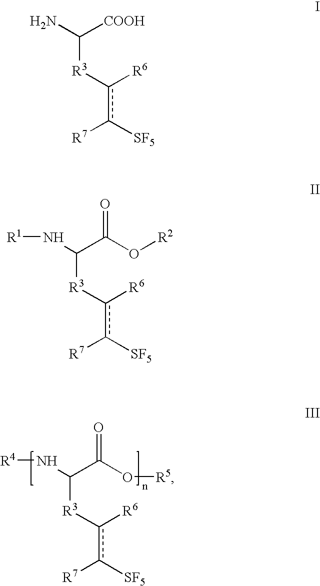
or a salt thereof, wherein 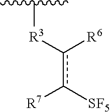
denotes a moiety selected from 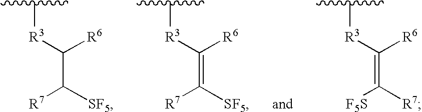
R¹ is chosen from H and an amino protecting group;
R² is chosen from H and C₁-C₆ alkyl;
R³ is independently at each occurrence chosen from C₁-C₆ alkylene and a direct bond;
R⁴ is chosen from H, an amino protecting group, an amino acid, a peptide, and a protein;
R⁵ is chosen from H, C₁-C₆ alkyl, an amino acid, a peptide, and a protein;
R⁶ is independently at each occurrence chosen from H and C₁-C₂ alkyl and
R⁷ is independently at each occurrence chosen from H and C₁-C₃ alkyl, and
n is an integer of 1 or more.

3. A compound according to claim 2 which is a compound of formula III wherein R⁴ is t-butoxycarbonyl.

4. A compound according to claim 2 wherein R³ is chosen from a direct bond, —CH₂—, —CH(CH₃)—, —CH(CH₃)—CH₂— and —CH₂—CH(CH₃)—.

5. A compound according to claim 2 wherein

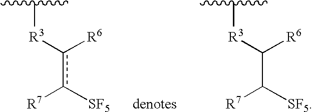

6. A compound according to claim 2 wherein

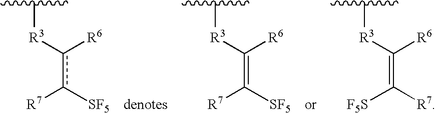

7. A compound according to claim 1 of formula IVa or IVb 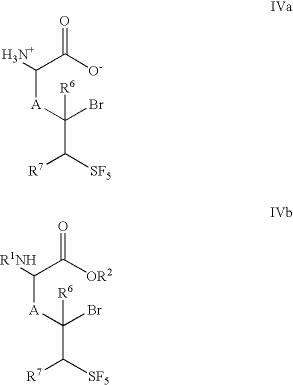
wherein
R¹ is chosen from H and an amino protecting group;
R² is chosen from H and C₁-C₆ alkyl;
A is chosen from C₁-C₅ hydrocarbon and a direct bond;
R⁶ is chosen from H and C₁-C₂ alkyl; and
R⁷ is chosen from H and C₁-C₃ alkyl.

8. A compound according to claim 1 which is selected from the group consisting of formulae Ia, IIa and IIIa: 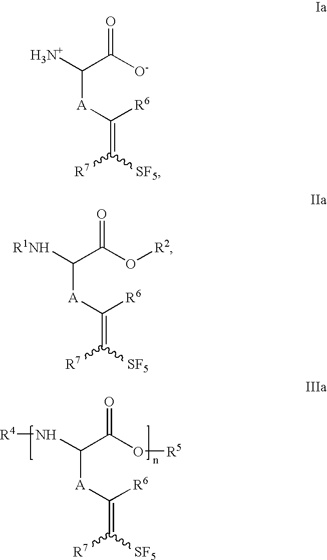
wherein
R¹ is chosen from H and an amino protecting group;
R² is chosen from H and C₁-C₆ alkyl;
A is independently at each occurrence chosen from C₁-C₅ hydrocarbon and a direct bond;
R⁴ is chosen from H, an amino protecting group, an amino acid, a peptide, and a protein;
R⁵ is chosen from H, C₁-C₆ alkyl, an amino acid, a peptide, and a protein;
R⁶ is independently at each occurrence chosen from H and C₁-C₂ alkyl
R⁷ is independently at each occurrence chosen from H and C₁-C₃ alkyl; and
n is an integer of 1 or more.

9. A compound according to claim 8 which is a compound of formula IIIa wherein R⁴ is t-butoxycarbonyl.

10. A compound according to claim 8 or claim 9 wherein A is chosen from a direct bond, —CH₂—, —CH(CH₃)—, —CH(CH₃)—CH₂— and —CH₂—CH(CH₃)—.

11. A process for SF₅X addition, wherein X is Br or Cl, comprising reacting a compound having an alkene functional group —CH═CH— with a solution of SF₅X in (a) one more or C₁-C₃ chlorofluorocarbons, (b) one or more C₅-C₆ alkyls, or (c) a mixture of at least one C₁-C₃ chlorofluorcarbon and at least one C₅-C₆ alkyl, where said reacting yields an SF₅X addition product

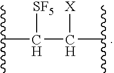

12. A process according to claim 11, wherein said compound having an alkene functional group is a compound of formula V: 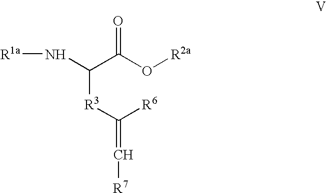
wherein
R^{1a} is an amino protecting group;
R^{2a} is C₁-C₆ alkyl;
R³ is chosen from C₁-C₆ alkylene and a direct bond;
R⁶ is chosen from H and C₁-C₂ alkyl;
R⁷ is chosen from H and C₁-C₃ alkyl;
X is Br; and
said SF₅X addition product is a compound of formula IV: 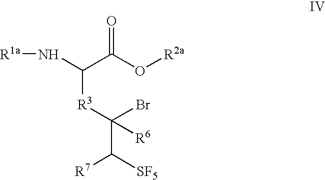
wherein R^{1a}, R^{2a}, R³, R⁶ and R⁷ are as defined above.

13. A process according to claim 11, wherein the solvent contains at least one C₁-C₃ chlorofluorocarbon.

14. A process according to claim 11, wherein the solvent is CCl₃F.

15. A process according to claim 11, wherein the solvent is a C₆ alkyl or a mixture of C₆ alkyls.

16. A compound of formula VIa, VIb, VIc or Vid: 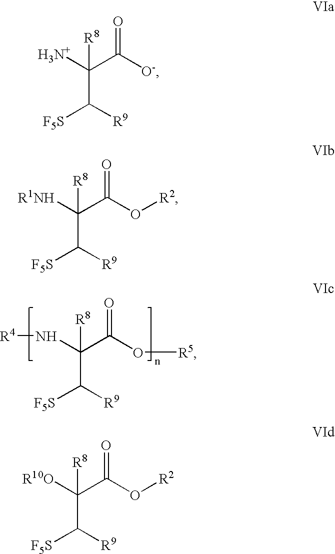
wherein
R¹ is chosen from H and an amino protecting group;
R² is chosen from H and C₁-C₆ alkyl;
R⁸ is independently at each occurrence chosen from H and C₁-C₃ alkyl;
R⁹ is independently at each occurrence chosen from —CH═CH₂ and —CH₂CH₃; and
R¹⁰ is chosen from H and an alcohol protecting group. In some embodiments, in the compound of formula VI, Y is NHBoc and Prot is trialkyl silyl.

17. A solution of SF₅X in a solvent selected from (a) one or more C₁-C₃ chlorofluorocarbons, (b) one or more C₅-C₆ alkyls, and (c) a mixture of at least one C₁-C₃ chlorofluorocarbons and at least one C₅-C₆ alkyls, wherein X is selected from Br and Cl.

18. (canceled)

19. (canceled)

20. The solution according to claim 17, wherein the solvent contains at least one C₁-C₃ chlorofluorocarbon.

21. (canceled)

22. (canceled)

23. (canceled)

24. (canceled)

25. A process for preparation of a compound of formula VI 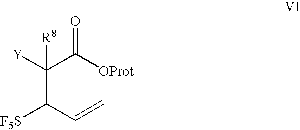
wherein R⁸ is H or alkyl, Prot is an enol protecting group, and Y is a protected amine, comprising reacting a compound of formula VII 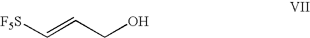
with a compound of formula VIII 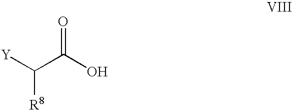
to obtain a compound of formula IX 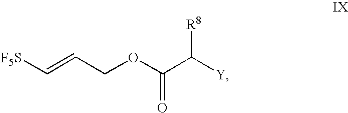
then reacting the compound formula IX with a strong, non-nucleophilic base, followed by reacting the resulting mixture with an enolate-reactive reagent for protecting an enol to obtain a compound of formula X 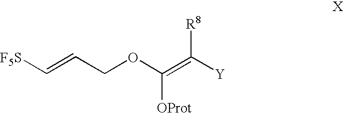
and exposing the compound of formula X to conditions in which the compound of formula X rearranges to form a compound of formula VI.

26. (canceled)

27. (canceled)

28. (canceled)

29. A compound of formula VI 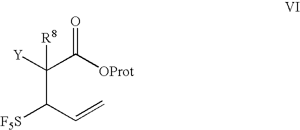
wherein R⁸ is H or alkyl, Prot is an enol protecting group, and Y is a protected amine.

30. (canceled)

31. (canceled)

32. (canceled)
